(19) United States
(12) Patent Application Publication
(10) Pub. No.: US 2025/0154160 A1
(43) Pub. Date: May 15, 2025

(54) METHOD OF MAKING BUPRENORPHINE AND PRECURSOR COMPOUNDS THEREOF

(71) Applicant: Arevipharma GmbH, Radebeul (DE)

(72) Inventors: Rostyslav Ukis, Coswig (DE); Heike Heydemüller, Dresden (DE); Kathrin Biskup, Dresden (DE); Michael Limmert, Dresden (DE)

(21) Appl. No.: 18/710,141

(22) PCT Filed: Dec. 15, 2022
(86) PCT No.: PCT/EP2022/086031

§ 371 Date: May 14, 2024

(30) Foreign Application Priority Data
Dec. 16, 2021 (EP) 21215313.4

(51) Int. Cl. C07D 491/08

(52) U.S. Cl. CPC C07D 491/08

(57) ABSTRACT

Method of making Buprenorphine comprising a Grignard reaction, comprising step (A): (A) performing the Grignard reaction in the presence of a compound of formula IIIa or IIIf R¹—[O—CHR²—CHR³]n-OR⁴ IIIa wherein n is an integer in the range of from 1 to 5; and (I) R¹ and R⁴ are different from one another and are C₁ to C₄ alkyl; and R² and R³ are independently H or C₁ to C₄ alkyl; or (II) R¹ and R⁴ are identical and are C₁ to C₄ alkyl; and R² and R³ are different from one another and are H or C₁ to C₄ alkyl; or (III) R¹ and R⁴ are identical and are C₂ to C₄ alkyl; and R² and R³ are independently H or C₁ to C₄ alkyl; or (IV) R¹ and R⁴ are independently from one another C₁ to C₄ alkyl; and R² and R³ taken together form a —(CH₂—CH₂—CH₂—[CH₂]m)- group, wherein m is an integer of from 0 to 2; or (V) IIIf wherein R¹ and R⁴ are defined as above with respect to compound IIIa, m is an integer in the range of 0 to 2, and x is 0 or 1.

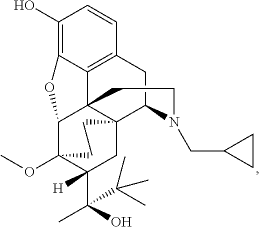 [SMILES: CO[C@]12CC[C@@]3(C[C@@H]1[C@](C)(O)C(C)(C)C)[C@H]1Cc4ccc(O)c5c4[C@@]3(CCN1CC1CC1)[C@H]2O5]

R¹—[O—CHR²—CHR³]ₙ—OR⁴ IIIa

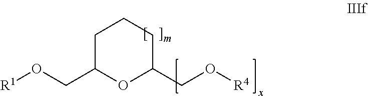

### FIELD OF THE INVENTION

The present invention relates to a synthesis of the K-opioid receptor antagonist Buprenorphine or a pharmaceutically acceptable salt thereof, respectively precursor compounds thereof, comprising a Grignard reaction.

### BACKGROUND OF THE INVENTION

Buprenorphine belongs to the class of semisynthetic opiates. It is medicinally used as an analgesic, indicated for the treatment of moderate to severe pain and opioid dependence. Usually, the starting point for the synthesis of Buprenorphine is thebaine. The most widely used process is known from GB 1 136 214 and citations therein. The synthetic sequence comprises the following stages depicted in Scheme 1:

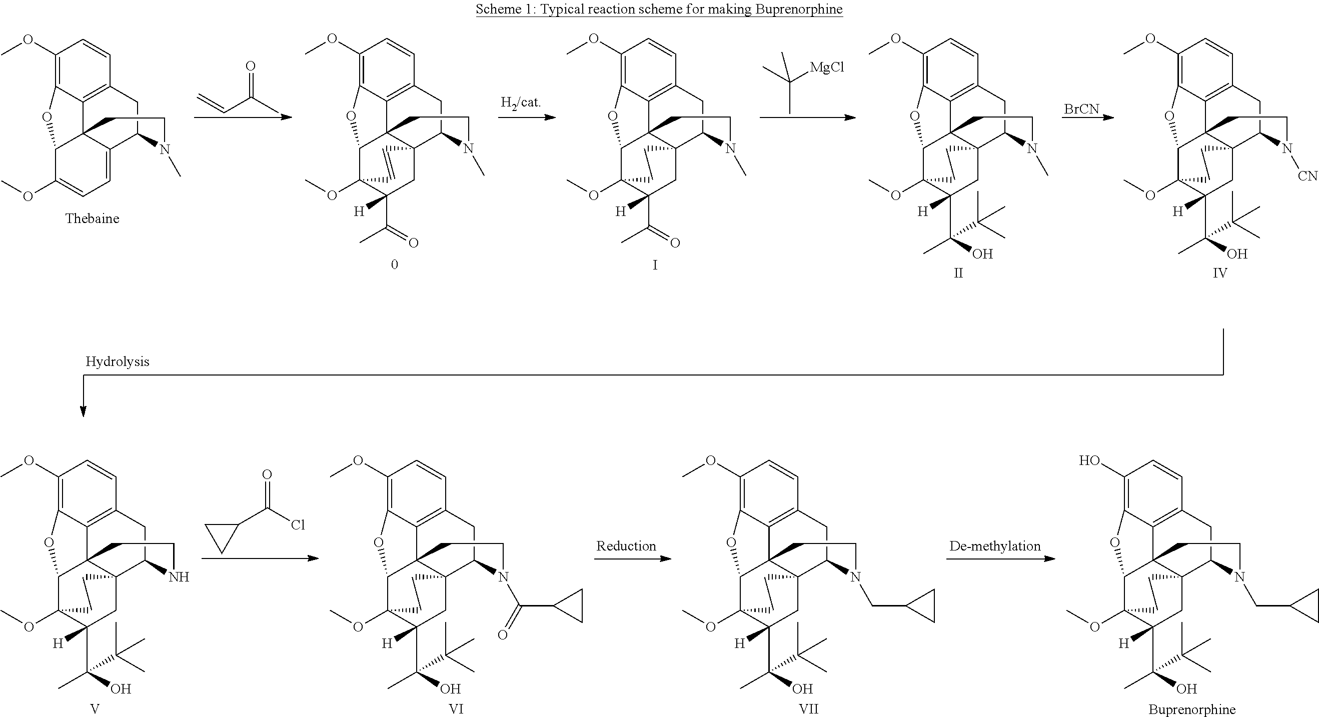

One of the precursor compounds of Buprenorphine is alcohol compound II which is obtained from compound I via alkylation of the acetyl group using t-buMgCl as Grignard reagent. Alternatively, instead of compound I, unsaturated compound 0 may be used which is subjected to the reaction with the Grignard reagent to afford a compound II′, followed by hydrogenation in one of the subsequent reaction steps:

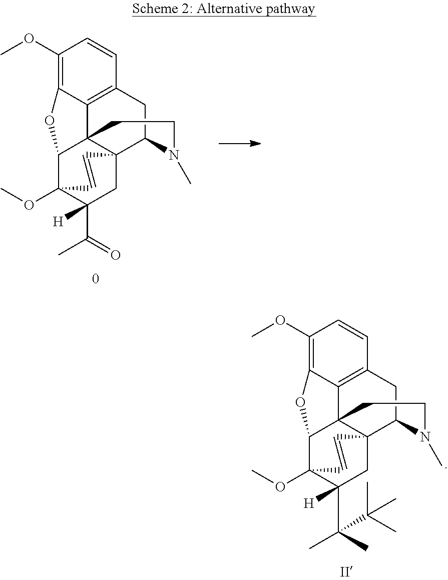

Deprotection and dealkylation stages often require forcing conditions and account for a significant generation of impurities. Thus, such stages have been subject of intensive investigation for instance as disclosed in patent specifications EP 2 344 507 (optimized O-dealkylation), EP 2 344 509 (single stage O-dealkylation and N-decyanation), EP 2 763 996 (improved deprotection) or EP 3 321 269 (optimized O-dealkylation).

Despite these advances there are still drawbacks. This pertains particularly to the addition reaction of the 7-acetyl-substitutent of the compound of formula I (or an analogue such as 0) with the Grignard reagent to afford the tertiary alcohol II or II′. This step is known to deliver mediocre yields and several by-products, most notable regenerated starting material from deprotonation and reduction products originating from hydride-transfer of the sterically hindered Grignard reagent.

The perennial desire to improve upon this key stage of the Buprenorphine manufacturing process can be exemplified with the following documents of the prior art as set out below:

The use of toluene or benzene as solvent in the Grignard reaction is supposed to minimize the undesirable side reactions and to improve turnover. However, the yield given was stated to be only ca. 25% of compound II after recrystallization (GB 1 136 214 using benzene as solvent).

The interaction of coordinating solvents or additives to fine-tune the reaction by blocking binding sites or enabling further cross-interactions at the magnesium center(s) is also well known to influence the Grignard reaction via coordination (e.g. with a polyether) or by steric shielding (Me-THF) or creating more dimeric species (addition of more readily available halide). The advantageous influence of polar/non-polar solvent mixtures was disclosed in WO 2010/039221. The yield was reported to be 55%.

Further attempts to improve the performance of this reaction stage included the execution of recycling stages (EP 2 342 208), thereby increasing the yield further from 55% to 72%. This, however, comes at the expense of significant labor effort and long cycle times per batch.

Particularly diglyme (diethyleneglycol dimethylether) as a solvent has its well-known application as (co-)solvent for Grignard addition reactions and is even referred to in this capacity by the European Chemicals Agency (“Background document for bis(2-methoxyethyl) ether (Diglyme, DEGDME)” as of 29 Nov. 2012).

WO 2021151908 discloses the use of diglyme as depicted in Scheme 3 below in addition to the polar solvent (as executed in Example “III. Reaction step b)” of said patent application). t-Butylmagnesium chloride was pre-mixed with diglyme, then the substrate of formula I was added as solution. This improved the yield further to ca. 80%, without the need for recycling stages:

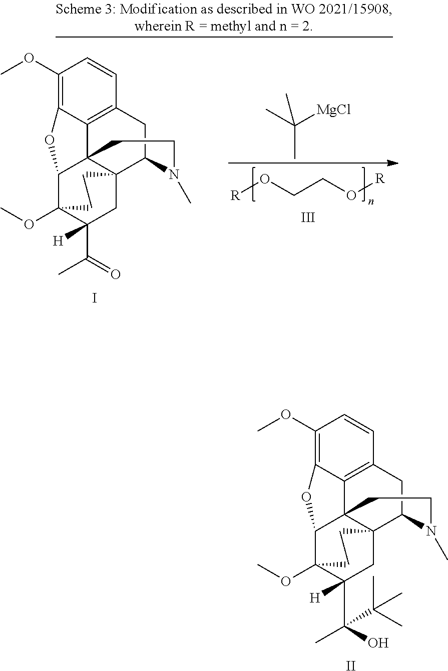

Despite this significant improvement for the manufacture of Buprenorphine, the use of diglyme renders the method of limited commercial use since the European Chemicals Agency (ECHA) marks diglyme as candidate for a substance of very high concern (SVHC). These substances may eventually be banned from usage and are expected to be substituted with a more benign alternative whenever possible.

EP 0 632 043 discloses the use of further ethers such as diethylene glycol dibutyl ether for making Grignard reagents.

U.S. Pat. No. 2,552,676 also discloses the use of further ethers such as diethylene glycol n-butyl ethyl ether and tetraethylene glycol dimethyl ether for making Grignard reagents.

### OBJECTS OF THE INVENTION

There is a need to improve upon the prior art and to deliver a process for the manufacture of Buprenorphine with a Grignard addition stage that maintains high yield and high purity, but also circumvents the usage of substances that are a hazard to human health.

### SUMMARY OF THE INVENTION

It was surprisingly discovered that diglyme used for performing a Grignard reaction in the synthesis of Buprenorphine could be substituted with readily available, non-toxic alternative compounds instead of the compound of formula III (R=methyl and n=2) with none or only little loss in efficiency.

Thus, the above object has been achieved with a method for making Buprenorphine, respectively precursor compounds II or II′, which employs an additive in the Grignard reaction that has the advantage to facilitate high yields whilst featuring a benign toxicological profile.

According to the invention, this process requires the use of O,O-dialkylated oligo ethylene oxides of the formula IIIa or of the formula IIIf

R¹—[O—CHR²—CHR³]ₙ—OR⁴ IIIa
wherein
n is an integer in the range of from 1 to 5, preferably 1 to 3, most preferred 2, and
(I) R¹ and R⁴ are different from one another and are C₁ to C₄ alkyl; and R² and R³ are independently H or C₁ to C₄ alkyl; or
(II) R¹ and R⁴ are identical and are C₁ to C₄ alkyl; and R² and R³ are different from one another and are H or C₁ to C₄ alkyl; or
(III) R¹ and R⁴ are identical and are C₂ to C₄ alkyl; and R² and R³ are independently H or C₁ to C₄ alkyl; or
(IV) R¹ and R⁴ are independently from one another C₁ to C₄ alkyl; and R² and R³ taken together form a —(CH₂—CH₂—CH₂—[CH₂]ₘ)— group, wherein m is an integer of from 0 to 2; or

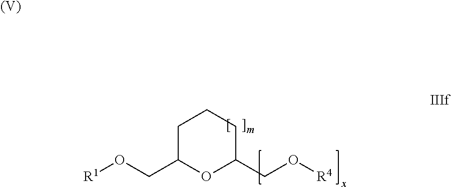

wherein R¹ and R⁴ are defined as above with respect to compound IIIa,
m is an integer in the range of 0 to 2, and x is 0 or 1.

Thus, compounds of formula IIIa

R¹—[O—CHR²—CHR³]ₙ—OR⁴ IIIa
in which R¹═R⁴═C₁ alkyl, R²═R³═H and n is an integer in the range of from 1 to 5 are not used in the method according to the invention. Accordingly, compounds where, e.g., n=1 (glyme), n=2 (diglyme), n=3 (triglyme), n=4 (tetraglyme) and n=5 (pentaglyme) are not used in the method according to the invention.

According to a first aspect, the invention relates to a method of making a compound of formula II or II′ as shown in Schemes 1, 2 and 3, wherein in the Grignard reaction a compound of formula IIIa or IIIf is used. Preferred embodiments are specified in the claims depending thereon.

According to a second aspect, the invention relates to a method of making Buprenorphine, the method comprising the method defined in the first aspect.

According to a third aspect, the invention relates to a method of performing a Grignard reaction, wherein a sterically hindered tertiary alkyl magnesium halide is reacted in the presence of a compound of formula IIIa or IIIf.

### DETAILED DESCRIPTION OF THE INVENTION

The method of performing the Grignard reaction as disclosed herein comprises step (A):

(A) performing the Grignard reaction in the presence of a compound of formula IIIa or IIIf

R¹—[O—CHR²—CHR³]ₙ—OR⁴ IIIa
wherein
n is an integer in the range of from 1 to 5; and
(I) R¹ and R⁴ are different from one another and are C₁ to C₄ alkyl; and R² and R³ are independently H or C₁ to C₄ alkyl; or
(II) R¹ and R⁴ are identical and are C₁ to C₄ alkyl; and R² and R³ are different from one another and are H or C₁ to C₄ alkyl; or
(III) R¹ and R⁴ are identical and are C₂ to C₄ alkyl; and R² and R³ are independently H or C₁ to C₄ alkyl; or
(IV) R¹ and R⁴ are independently from one another C₁ to C₄ alkyl; and R² and R³ taken together form a —(CH₂—CH₂—CH₂—[CH₂]ₘ)— group, wherein m is an integer of from 0 to 2; or

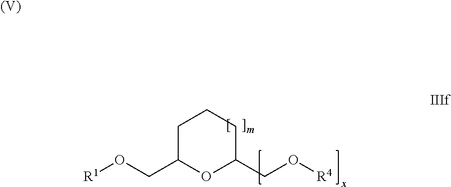

wherein R¹ and R⁴ are defined as above with respect to compound IIIa,
m is an integer in the range of 0 to 2, and x is 0 or 1.

In a preferred embodiment, n is an integer in the range of from 1 to 3.

In one embodiment, n is 1 or 2 or 3.

In a particularly preferred embodiment, n is 2.

The presence of a compound of formula IIIa increases the selectivity towards the desired addition reaction of the Grignard reagent used in step (A) and/or increases the reaction rate, and/or increases the yield compared to a reaction, which is void of compound IIIa, respectively.

As used herein, the term “Grignard reaction” is used in the commonly accepted meaning of an organometallic chemical reaction in which alkyl, allyl, vinyl, or aryl-magnesium halides, i.e., the Grignard reagent, react preferably with a carbonyl group in an aldehyde or ketone upon forming a carbon-carbon bond.

According to the invention, the Grignard reagent used in the Grignard reaction according to step (A) comprises a t-butylMgX compound, wherein X is chloride, bromide or iodide.

The compounds of formula IIIa are known or may be produced according to known methods.

The term “C₁ to C₄ alkyl” as used in connection with the compound of formula IIIa encompasses the individual C₁, C₂, C₃ and C₄ alkyls.

As used herein, the term “C₁ alkyl” means CH₃.

As used herein, the term “C₂ alkyl” means C₂H₅.

As used herein, the term “C₃ alkyl” means C₃H₇, either linear or branched, i.e., n-propyl or iso-propyl.

As used herein, the term “C₄ alkyl” means C₄H₉, i.e., linear butyl and the various branched butyls.

In a particularly preferred embodiment, the compound of formula IIIa comprises or is a compound of formula IIIb, IIIc, IIId, or IIIe:

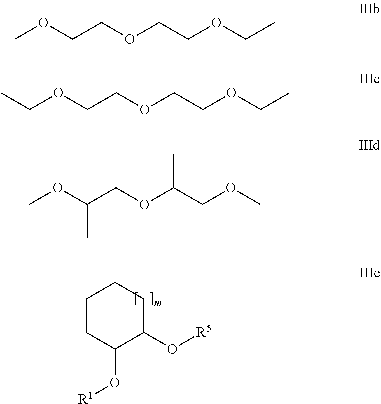

Compound IIIb is diethylene glycol methyl ethyl ether (CAS no. 1002-67-1).

Compound IIIc is diethylene glycol diethyl ether (CAS no. 112-36-7).

Compound IIId is dipropylene glycol dimethyl ether (CAS no. 111109-77-4). The compound may comprise a mixture of stereo- and regioisomers.

In one embodiment, R² and R³ of compound IIIa can be connected to form a cyclic structure.

In another embodiment, R² and R³ in compound IIIa originate from the same ethylene moiety, thus forming a 1,2-dialkoxy substituted cyclic structure resulting in a compound of formula IIIe.

Herein, R¹ and R⁵ in IIIe may be defined as above wherein either R⁵ equals R⁴, or R⁵ is an alkoxy-ethylene-group of the type —[CHR²—CHR³—OR⁴], where R², R³ and R⁴ are defined as above. m is an integer in the range of 0 to 2. Compound IIIe may comprise a mixture of diastereomers.

In one embodiment, herein referred to a compound IIIf with the following formula

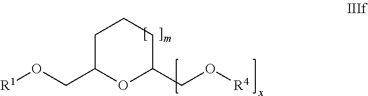

R¹ and R⁴ are defined as above and m is an integer in the range of 0 to 2 and x is 0 or 1. In one embodiment of compound IIIf, both R¹ and R⁴ can be methyl. Compound IIIf may comprise a mixture of stereoisomers.

A suitable compound of formula IIIf is e.g.

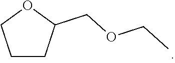

The compound is known (CAS no. 62435-76-6).

In a further preferred embodiment, the Grignard reaction according to step (A) is performed in the presence of the compound of formula IIIa or IIIf and a solvent.

Preferably, the solvent is selected from an ether, a hydrocarbon and an aromatic hydrocarbon, or a mixture of two or three thereof. Thus, the solvent is or may comprise a mixture of an ether with a hydrocarbon, or a mixture of an ether with an aromatic hydrocarbon, or a mixture of a hydrocarbon with an aromatic hydrocarbon.

In a preferred embodiment, the solvent comprises or is a mixture of an ether with an aromatic hydrocarbon.

In one embodiment, the ether is selected from the group consisting of diethyl ether, methyl t-butyl ether, tetrahydrofuran, 2-methyltetrahydrofuran and anisole.

In one embodiment, the aromatic hydrocarbon is selected from benzene, toluene, xylene and mesitylene.

In a preferred embodiment, the compound of formula IIIa or IIIf is used in a solvent comprising or is a solvent consisting of a mixture of an ether, preferably diethyl ether, and an aromatic hydrocarbon, preferably toluene or benzene, particularly preferred toluene.

Step (A) further comprises a carbonyl compound to subject the carbonyl compound to alkylation using the Grignard reagent.

According to the invention, the carbonyl compound is either a compound of formula I or 0.

Thus, the invention relates to a method of making a compound of formula II from a compound of formula I or a compound of formula II′ from a compound of formula 0, the method comprising step (A):
(A) reacting the compound of formula I or 0 with a Grignard reagent t-butylMgX in the presence of a compound of formula IIIa or IIIf, as respectively defined above.
and
X=chloride, bromide or iodide.

In a particularly preferred embodiment, step (A) comprises steps (a) and (b):
(a) adding a compound of formula IIIa or IIIf to a solution of the Grignard reagent in a solvent as defined above;
(b) adding the carbonyl compound of formula I or 0 to the solution defined in step (a)
(c) optionally stirring the reaction mixture obtained in step (b);
(d) terminating the reaction;
(e) isolating and optionally purifying the reaction product of the Grignard reagent with the carbonyl compound.

In a particularly preferred embodiment, the method comprises
(a) adding a compound of formula IIIa or IIIf to an ethereal solution of t-butylmagnesium halide with toluene;
(b) adding the carbonyl compound of formula I or 0 to the solution defined in step (a) whilst stirring at a temperature at or above ambient temperature;
(c) optionally stirring the reaction mixture obtained in step (b), preferably for less than 1 h;
(d) terminating the reaction;
(e) isolating and optionally purifying the product of the Grignard compound with the carbonyl compound.

The addition period of the carbonyl compound to the solution defined in step (b) is not particularly limited. However, since the reaction of the carbonyl compound I or 0 with the Grignard reagent typically proceeds instantaneously, the addition period can be short, minimizing thermal stress. This short addition period is particularly advantageous in case of polycyclic carbonyl compounds such as compound 0 which can potentially rearrange under the employed conditions (K. W. Bentley et al., J. Am. Chem. Soc. 1967, 89 (13), 3312-3321).

Accordingly, the addition of the carbonyl compound can range from instantaneous to 2 h, preferably from instantaneous to 30 min.

In a preferred embodiment, the temperature employed in step (b) is in the range of from 20 to 100° C., preferably 30 to 80° C., further preferred 40 to 80° C., still further preferred 50 to 70° C., and most preferred approx. 60° C.

The stirring period used in step (c) is not particularly limited. It may range from 0 min to 2 h, preferably 15 min to 2 h.

The termination of the reaction may be effected by commonly known methods such as decomposing the magnesium complexes formed in the Grignard reaction with water, preferably in the presence of an ammonium salt.

Subsequently, the formed product may be isolated by filtration or may be extracted, optionally followed by commonly known purification methods such as re-crystallization or distillation.

The process is rather stable and allows for a rather broad range of parameter variations whilst maintaining good to excellent turnover.

### Second Aspect: Method of Making Buprenorphine or a Pharmaceutically Acceptable Salt Thereof

As shown above, the method as defined in the first aspect may be advantageously used in the synthesis of precursor compounds II or II′ of Buprenorphine.

Thus, according to a second aspect, the invention relates to a method of making Buprenorphine or a pharmaceutically acceptable salt thereof comprising a method as defined in the first aspect.

### Third Aspect

As shown in the first aspect, the method according to the invention is particularly beneficial for performing a Grignard reaction of a sterically hindered Grignard reagent such as t-buMgX.

Thus, according to a third aspect, the invention relates to a method of performing a Grignard reaction, the method comprising step (A):
(A) performing the Grignard reaction of a Grignard reagent t-alkylMgX in the presence of a compound of formula IIIa or IIIf

R¹—[O—CHR²—CHR³]ₙ—OR⁴ IIIa
wherein
n is an integer in the range of from 1 to 5; and
(I) R¹ and R⁴ are different from one another and are C₁ to C₄ alkyl; and R² and R³ are independently H or C₁ to C₄ alkyl; or
(II) R¹ and R⁴ are identical and are C₁ to C₄ alkyl; and R² and R³ are different from one another and are H or C₁ to C₄ alkyl; or
(III) R¹ and R⁴ are identical and are C₂ to C₄ alkyl; and R² and R³ are independently H or C₁ to C₄ alkyl; or
(IV) R¹ and R⁴ are independently from one another C₁ to C₄ alkyl; and R² and R³ taken together form a —(CH₂—CH₂—CH₂—[CH₂]ₘ)— group, wherein m is an integer of from 0 to 2; or

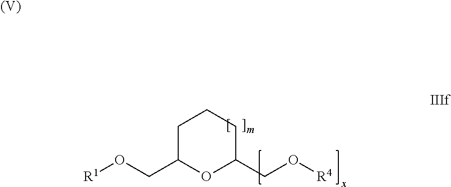

wherein R¹ and R⁴ are defined as above with respect to compound IIIa,
m is an integer in the range of 0 to 2, and x is 0 or 1.
wherein X is chloride, bromide or iodide; and
t-alkyl is a tertiary alkyl group having from 4 to 10 carbon atoms.

In a preferred embodiment, t-alkyl is t-butyl.

### EXAMPLES

In the following, the methods according to the invention are exemplified as follows:

### Example 1: Ligand According to Structure IIIb

To a mixture of 146 mL of toluene and 175 ml of tert-butyl magnesium chloride (25% in diethylether) 18 g of diethylene glycol methyl ethyl ether (DEGMEE) was added within 5 to 10 min at T≤35° C. The suspension was heated to T=40° C. and a solution of 30.0 g of compound of formula I in 114 mL of warm toluene was added within 30 min whilst maintaining a temperature of around 60° C. Then the suspension was stirred for 15 min at this temperature. An HPLC aliquot was taken for analysis. HPLC (relative area percentage): 90% of compound of formula II.

After cooling to ambient temperature, a saturated, aqueous solution of ammonium chloride was added. The resulting two phases were separated; warming up slightly may improve separation. The product containing organic phase was extracted with water.

From the organic product phase, the solvent was distilled off under reduced pressure. Two consecutive times, ethanol was added and distilled off under reduced pressure. Then 12 mL of ethanol were added, the mixture was heated to reflux until full dissolution and 3 ml of water were added. A suspension ensued which was stirred briefly, then cooled slowly to ambient temperature and stirred for 2 h. Finally, the product was filtered off, washed with ethanol-water 1:1 and dried.

Yield: 25.9 to 29.4 g (75-85%) of compound of formula II.

### Comparative Example 1: Bidentate Ligand, n=1: Glyme

To a mixture of 49.0 mL of toluene and 58.3 mL of tert-butyl magnesium chloride (25% in diethylether) 3.6 g of 1,2-dimethoxyethane (glyme) was added within 5 to 10 min at T≤35° C. The yellow solution was heated to reflux and stirred for 15 min. A solution of 10.0 g of compound of formula I in 38.0 mL of warm toluene was added within 30 min whilst maintaining a temperature of around 60° C. Then the suspension was stirred for 15 min at this temperature. An HPLC aliquot was taken for analysis. HPLC (relative area percentage): 82-90% of compound of formula II.

After cooling to ambient temperature, a saturated, aqueous solution of ammonium chloride was added. The resulting two phases were separated; warming up slightly may improve separation. The product containing organic phase was extracted with water.

From the organic product phase, the solvent was distilled off under reduced pressure. Two consecutive times, ethanol was added and distilled off under reduced pressure. Then 46.3 mL of ethanol were added, the mixture was heated to reflux until full dissolution and 11.8 mL of water were added. A suspension ensued which was stirred briefly, then cooled slowly to ambient temperature and stirred for 18 h. Finally, the product was filtered off, washed with ethanol-water 4:1 and dried.

Yield: 8.98 (78%) of compound of formula II.

### Example 2: Ligand According to Structure IIIc

To a mixture of 49.0 mL of toluene and 58.3 mL of tert-butyl magnesium chloride (25% in diethylether) 6.6 g of dipropylenglycoldimethylether (DPGDME) was added within 5 to 10 min at T≤35° C. The yellow solution was heated to 40° C. and stirred for 15 min. A solution of 10.0 g of compound of formula I in 38.0 mL of warm toluene was added within 30 min whilst maintaining a temperature of around 60° C. Then the suspension was stirred for 15 min at this temperature. An HPLC aliquot was taken for analysis. HPLC (relative area percentage): 83% of compound of formula II.

After cooling to ambient temperature, a saturated, aqueous solution of ammonium chloride was added. The resulting two phases were separated; warming up slightly may improve separation. The product containing organic phase was extracted with water.

From the organic product phase, the solvent was distilled off under reduced pressure. Two consecutive times, ethanol was added and distilled off under reduced pressure. Then 40 mL of ethanol were added, the mixture was heated to reflux until full dissolution and 10 ml of water were added. A suspension ensued which was stirred briefly, then cooled slowly to ambient temperature and stirred for 2 h. Finally, the product was filtered off, washed with ethanol-water 1:1 and dried.

Yield: 8.75 g (76%) of compound of formula II.

### Example 3: Ligand According to Structure IIId

To a mixture of 9.7 mL of toluene and 11.7 mL of tert-butyl magnesium chloride (25% in diethylether) 1.3 g of diethyleneglycoldiethylether was added within 5 to 10 min at T≤35° C. The yellow solution was heated to 40° C. and stirred for 15 min. A solution of 2.0 g of compound of formula I in 7.6 mL of warm toluene was added within 30 min whilst maintaining a temperature of around 60° C. Then the suspension was stirred for 15 min at this temperature. An HPLC aliquot was taken for analysis.

HPLC (relative area percentage): 82% of compound of formula II. The reaction was not worked up since the IPC adequately reflected the expected yield, as was demonstrated in examples 1 and 2 and as well in comparative example 1.

### Example 4: Ligand According to Structure IIIf

To a mixture of 9.7 mL of toluene and 11.7 mL of tert-butyl magnesium chloride (25% in diethylether) 1.3 g of 2-(ethoxymethyl)tetrahydrofuran was added within 5 to 10 min at T≤35° C. The yellow solution was heated to 40° C. and stirred for 15 min. A solution of 2.0 g of compound of formula I in 7.6 mL of warm toluene was added within 30 min whilst maintaining a temperature of around 60° C. Then the suspension was stirred for 15 min at this temperature. An HPLC aliquot was taken for analysis.

HPLC (relative area percentage): 82% of compound of formula II. The reaction was not worked up since the IPC adequately reflected the expected yield, as was demonstrated in examples 1 and 2 and as well in comparative example 1.

1. A method of making a compound of formula II from a compound of formula I 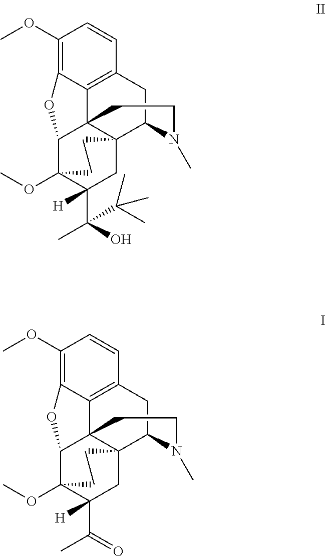
or a compound of formula II′ from a compound of formula 0 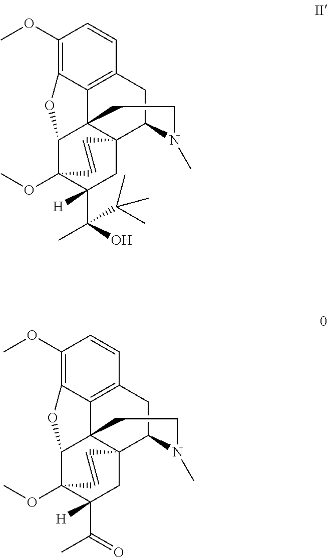
the method comprising step (A):
(A) reacting the compound of formula I or 0 with a Grignard reagent t-butylMgX, wherein X is chloride, bromide or iodide, in the presence of a compound of formula IIIa or IIIf
R¹—[O—CHR²—CHR³]ₙ—OR⁴ IIIa
wherein
n is an integer in the range of from 1 to 5; and
(I) R¹ and R⁴ are different from one another and are C₁ to C₄ alkyl; and R² and R³ are independently H or C₁ to C₄ alkyl; or
(II) R¹ and R⁴ are identical and are C₁ to C₄ alkyl; and R² and R³ are different from one another and are H or C₁ to C₄ alkyl; or
(III) R¹ and R⁴ are identical and are C₂ to C₄ alkyl; and R² and R³ are independently H or C₁ to C₄ alkyl; or
(IV) R¹ and R⁴ are independently from one another C₁ to C₄ alkyl; and R² and R³ taken together form a —(CH₂—CH₂—CH₂—[CH₂]ₘ)— group, wherein m is an integer of from 0 to 2; or 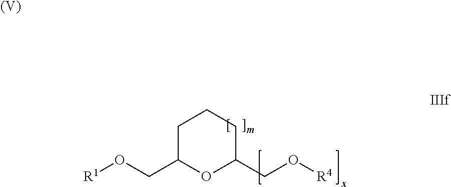
wherein R¹ and R⁴ are defined as above with respect to compound IIIa,
m is an integer in the range of 0 to 2, and x is 0 or 1.

2. The method of claim 1, wherein the compound of formula IIIa comprises or is a compound of formula IIIb, IIIc, IIId, IIIe: 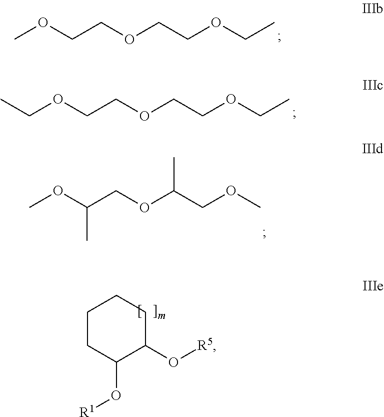
wherein R⁵═R⁴, and R¹ and R⁴ are defined as above with respect to compound IIIa, or
R⁵ is an alkoxy-ethylene-group of the type-[CHR²—CHR³—OR⁴], where R², R³ and R⁴ are defined as above with respect to compound IIIa;
m is an integer in the range of 0 to 2.

3. The method of claim 1, wherein in step (A) the reaction is performed in the presence of the compound of formula IIIa or IIIf and a solvent, wherein the solvent is selected from an ether, a hydrocarbon and an aromatic hydrocarbon, or a mixture of two or three thereof.

4. The method of claim 3, wherein the ether is selected from the group consisting of diethyl ether, methyl t-butyl ether, tetrahydrofuran, 2-methyltetrahydrofuran and anisole.

5. The method of claim 3, wherein the aromatic hydrocarbon is selected from toluene or benzene.

6. The method of claim 3,
wherein the solvent comprises or is a mixture of diethyl ether and toluene.

7. The method of claim 3, wherein step (A) comprises
(a) adding a compound of formula IIIa or IIIf to a solution of the Grignard reagent in the solvent;
(b) adding a compound of formula I or 0 to the solution defined in step (a).

8. The method of claim 7, comprising:
(a) adding a compound of formula IIIa or IIIf to an ethereal solution of t-butyl magnesium halide and toluene;
(b) adding the compound of formula I or 0 to the solution defined in step (a) whilst stirring at a temperature at or above ambient temperature;
(c) stirring the reaction mixture, preferably for less than 1 h;
(d) terminating the reaction.

9. The method of claim 8, wherein the temperature in step (b) is in the range of from 20 to 100° C., preferably 30 to 80° C.

10. A method of making Buprenorphine 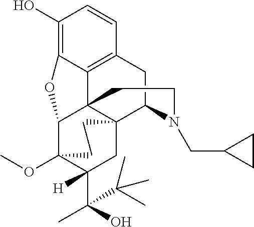 [SMILES: CO[C@]12CC[C@@]3(C[C@@H]1[C@](C)(O)C(C)(C)C)[C@H]1Cc4ccc(O)c5c4[C@@]3(CCN1CC1CC1)[C@H]2O5]
or a pharmaceutically acceptable salt thereof, the method comprising a method as defined in claim 1.

11. A method of performing a Grignard reaction, the method comprising step (A):
(A) performing the Grignard reaction of a Grignard reagent t-alkylMgX in the presence of a compound of formula IIIa or IIIf
R¹—[O—CHR²—CHR³]ₙ—OR⁴ IIIa
wherein
n is an integer in the range of from 1 to 5; and
(I) R¹ and R⁴ are different from one another and are C₁ to C₄ alkyl; and R² and R³ are independently H or C₁ to C₄ alkyl; or
(II) R¹ and R⁴ are identical and are C₁ to C₄ alkyl; and R² and R³ are different from one another and are H or C₁ to C₄ alkyl; or
(III) R¹ and R⁴ are identical and are C₂ to C₄ alkyl; and R² and R³ are independently H or C₁ to C₄ alkyl; or
(IV) R¹ and R⁴ are independently from one another C₁ to C₄ alkyl; and R² and R³ taken together form a —(CH₂—CH₂—CH₂—[CH₂]ₘ)— group, wherein m is an integer of from 0 to 2; or 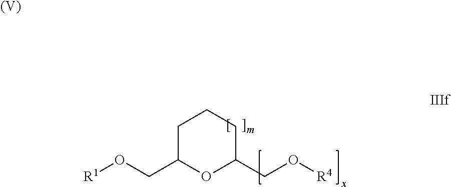
wherein R¹ and R⁴ are defined as above with respect to compound IIIa,
m is an integer in the range of 0 to 2, and x is 0 or 1;
wherein X is chloride, bromide or iodide; and
t-alkyl is a tertiary alkyl group having from 4 to 10 carbon atoms.

12. The method of claim 11, wherein t-alkyl is t-butyl.

13. The method according to claim 1, wherein the compound IIIf comprises or is [chemical structure]
